(19) United States
(12) Patent Application Publication
(10) Pub. No.: US 2023/0219913 A1
(43) Pub. Date: Jul. 13, 2023

(54) Sulfamoylamide Derivatives

(71) Applicant: Hoffmann-La Roche Inc., Little Falls, NJ (US)

(72) Inventors: Stefan BERCHTOLD, Binningen (CH); Guido GALLEY, Rheinfelden (CH); Katrin GROEBKE ZBINDEN, Liestal (CH); Wolfgang GUBA, Basel (CH); Danny KRUMM, Basel (CH)

(73) Assignee: Hoffmann-La Roche Inc., Little Falls, NJ (US)

(21) Appl. No.: 17/996,328

(22) PCT Filed: Apr. 14, 2021
(86) PCT No.: PCT/EP2021/059601

§ 371 Date: Oct. 14, 2022

(30) Foreign Application Priority Data
Apr. 16, 2020 (EP) 20169767.9

(51) Int. Cl. C07D 285/16; C07D 417/06

(52) U.S. Cl. CPC C07D 285/16; C07D 417/06

(57) ABSTRACT

The invention relates to a compound of formula (I) wherein R1-R3 are as defined in the description and in the claims. The compound of formula (I) can be used as a medicament.

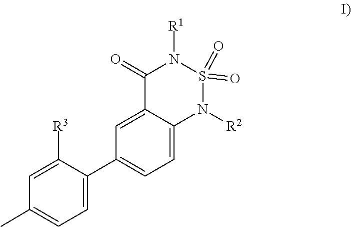

The present invention relates to organic compounds useful for therapy and/or prophylaxis in a mammal, and in particular to compounds that modulate cGAS activity.

The invention relates in particular to a compound of formula (I)

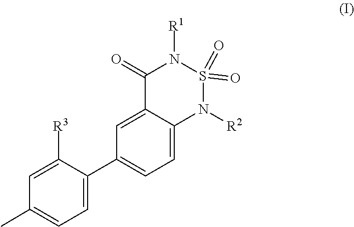

wherein
one of R¹ and R² is hydrogen and the other one is hydrogen, alkyl, morpholinylalkyl, pyridinylalkyl or phenylalkyl; and
R³ is halogen;
or a pharmaceutically acceptable salt thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production of Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trex1, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFα and IL1β.

As of today many diseases caused by dysregulation of the innate immune system lack efficient therapies.

The compound of the invention binds to and modulates cGAS activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, isopropyl, butyl, isobutyl, tert.-butyl and pentyl. Methyl and ethyl are particular examples of “alkyl” in the compound of formula (I).

The terms “halogen” or “halo”, alone or in combination, signifies fluorine, chlorine, bromine or iodine and particularly fluorine, chlorine or bromine, more particularly chlorine. The term “halo”, in combination with another group, denotes the substitution of said group with at least one halogen, particularly substituted with one to five halogens, particularly one to four halogens, i.e. one, two, three or four halogens.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of the compound of formula (I) are the salts of hydrochloric acid, hydrobromic acid, sulfuric acid, phosphoric acid and methanesulfonic acid.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

The invention thus relates to:

A compound according to the invention wherein one of R¹ and R² is hydrogen and the other one is hydrogen, alkyl, morpholinylethyl, pyridinylmethyl, phenylmethyl or phenyl ethyl;

A compound according to the invention wherein one of R¹ and R² is hydrogen and the other one is hydrogen or morpholinylalkyl;

A compound according to the invention wherein one of R¹ and R² is hydrogen and the other one is hydrogen or morpholinylethyl;

A compound according to the invention wherein R³ is chlorine;

A compound of formula (I) according to the invention selected from
6-(2-chloro-4-methylphenyl)-1H-benzo[c][1,2,6]thiadiazin-4(3H)-one 2,2-dioxide;
6-(2-chloro-4-methylphenyl)-1-(2-morpholinoethyl)-1H-benzo[c][1,2,6]thiadiazin-4(3H)-one 2,2-dioxide;
6-(2-chloro-4-methylphenyl)-1-(pyridin-4-ylmethyl)-1H-benzo[c][1,2,6]thiadiazin-4(3H)-one 2,2-dioxide;
6-(2-chloro-4-methylphenyl)-1-phenethyl-1H-benzo[c][1,2,6]thiadiazin-4(3H)-one 2,2-dioxide;
6-(2-chloro-4-methylphenyl)-3-(2-morpholinoethyl)-1H-benzo[c][1,2,6]thiadiazin-4(3H)-one 2,2-dioxide;
1-benzyl-6-(2-chloro-4-methylphenyl)-1H-benzo[c][1,2,6]thiadiazin-4(3H)-one 2,2-dioxide;
   or a pharmaceutically acceptable salt thereof.

A compound according to the selection from
6-(2-chloro-4-methylphenyl)-1H-benzo[c][1,2,6]thiadiazin-4(3H)-one 2,2-dioxide;
6-(2-chloro-4-methylphenyl)-1-(2-morpholinoethyl)-1H-benzo[c][1,2,6]thiadiazin-4(3H)-one 2,2-dioxide;
   or a pharmaceutically acceptable salt thereof.

The synthesis of the compound of formula (I) can, for example, be accomplished according to the following scheme, wherein the compound of formula (I) can be I, I-1 or I-2. Unless otherwise indicated, le-le have the meaning as defined above.

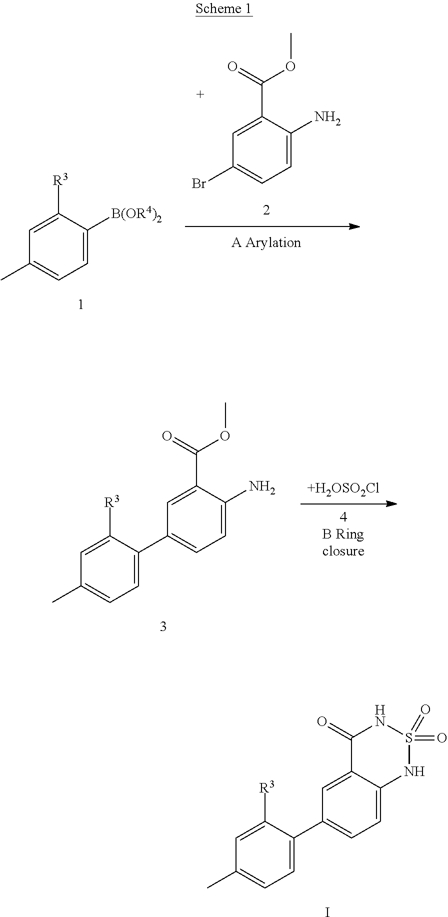

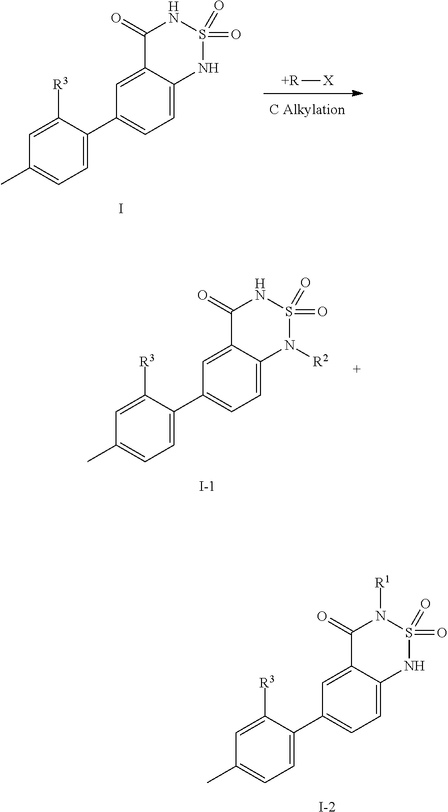

In scheme 1, R⁴ is hydrogen or alkyl.

In scheme 1, R³ is conveniently chloride.

Step A: Coupling of the bromoderivative 2 with the boronic acid or boronic acid ester 1 can be accomplished by using a palladium catalyst such as palladium(II)-acetate, palladium(II)-chloride, 1,1′-bis(diphenylphosphino)ferrocene-palladium(II)dichloride dichloromethane complex, tris(dibenzylideneacetone)dipalladium, tris(dibenzylideneacetone)dipalladium-chloroform adduct, or tetrakis(triphenylphosphine)palladium(0) in combination with a ligand such as triphenylphosphine, tricyclohexylphosphine, X-phos, Xantphos or the like, and a base such as potassium phosphate, potassium carbonate, cesium carbonate, triethylamine or diisopropylethylamine in a suitable solvent such as dioxane, toluene, dimethylacetamide, dimethylformamide, tetrahydrofuran, dimethoxyethane, diglyme, ethanol, methanol, water or mixtures of the solvents mentioned above at 20° C. to 180° C. for 5 min to 18 hrs with or without microwave irradiation.

Convenient conditions are the use of tris(dibenzylideneacetone)dipalladium-chloroform adduct, X-phos and potassium phosphate in a mixture of dioxane and water at 100° C. for 2-4 h.

Step B: Ring closure to form the cyclic sulfamoylamide I can be accomplished by reacting compound 3 with sulfamoyl chloride in a suitable solvent such as tetrahydrofuran, toluene or dioxane, followed by addition of an aqueous base such as sodium hydroxide or potassium hydroxide solution.

Convenient conditions are the use of sulfamoyl chloride in tetrahydrofuran at 25° C. for 5 h followed by addition of aqueous sodium hydroxide solution.

Step C: The alkylation/benzylation can be accomplished by reacting the cyclic sulfamoylamide I with a suitable substituted alkyl halide R—X such as substituted alkyl chlorides, substituted alkyl bromides, substituted alkyl iodides, or with a suitable substituted benzyl halide R—X, such as substituted benzyl bromides, substituted benzyl chlorides. The alkylation/benzylation can be performed with or without a base such as sodium bicarbonate, cesium carbonate, potassium carbonate, sodium carbonate, triethylamine or ethyldiisopropylamine in a solvent such as as water, dimethylacetamide, dimethylformamide or tetrahydrofuran at 0° C.-150° C. for 1 h to 18 h. If regioisomeric mixtures of alkylation products are obtained they can be separated by column chromatography on silica gel using mixtures of organic solvents such as heptane, ethylacetate, methanol and dichloromethane to yield the described regioisomer as a pure compound.

Preferred conditions are the use of substituted alkyl bromides or chlorides and aqueous sodium bicarbonate solution at 100° C. for several hours followed by chromatographic separation from the regioisomer.

The invention thus also relates to a process for the preparation of a compound according to the invention comprising one of the following steps:

(a) the reaction of a compound of formula (A)

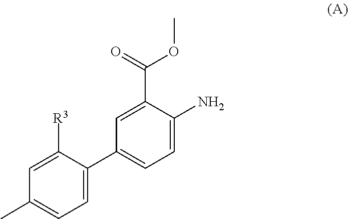

with H₂NSO₂Cl in the presence of a base wherein R³ is as defined above; or

(b) the reaction of a compound of formula (B)

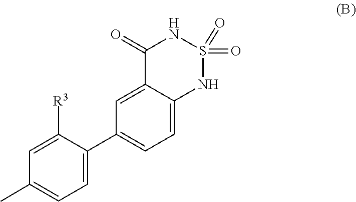

with R—X and optionally with a base wherein R³ is as defined above and wherein R is R¹ or R² as defined above and X is a leaving group.

The solvent of step (a) can advantageously be tetrahydrofuran, toluene or dioxane.

The base of step (a) can advantageously be sodium hydroxide or potassium hydroxide.

Preferred conditions for step (a) are the use of sulfamoyl chloride in tetrahydrofuran, in particular at 25° C. for 5 h, followed by the addition of an aqueous sodium hydroxide solution.

In step (b) the leaving group X is conveniently a halogen, a mesylate or a tosylate, in particular a halogen or a tosylate.

The solvent of step (b) can advantageously be water, dimethylacetamide, dimethylformamide or tetrahydrofuran.

The base of step (b) can advantageously be sodium bicarbonate, cesium carbonate, potassium carbonate, sodium carbonate, triethylamine or ethyldiisopropylamine.

Preferred conditions for step (b) are X=bromine or chlorine, aqueous sodium bicarbonate solution as a base, at 0° C.-150° C. for 1 h to 18 h, in particular at 100° C.

The regioisomers (I-1 and I-2 as defined above) can be separated by chromatography.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:

A compound of formula (I) for use in the treatment of a disease modulated by cGAS;

The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and

A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or

Aicardi-Goutieres syndrome (AGS), which method comprises administering an effective amount of a compound of formula (I) to a patient in need thereof.

The invention will now be illustrated by the following examples which have no limiting character.

### EXAMPLES

### Abbreviations

DMF=dimethylformamide; DMSO=dimethyl sulfoxide; ESI=electrospray ionization; EtOAc=ethyl acetate; HPLC=high performance liquid chromatography; MS=mass spectrometry; RT=room temperature.

### Example 1

### 6-(2-Chloro-4-methylphenyl)-1H-benzo[c][1,2,6]thiadiazin-4(3H)-one 2,2-dioxide

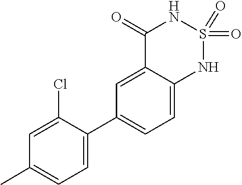 [SMILES: Cc1ccc(-c2ccc3c(c2)C(=O)NS(=O)(=O)N3)c(Cl)c1]

### a) 4-Amino-2′-chloro-4′-methyl-[1,1′-biphenyl]-3-carboxylate

Methyl 2-amino-5-bromobenzoate (5 g, 21.7 mmol, Eq: 1), (2-chloro-4-methylphenyl)boronic acid (3.7 g, 21.7 mmol, Eq: 1) and potassium phosphate (9.23 g, 43.5 mmol, Eq: 2) were combined with dioxane (80 ml) and water (20 ml). The vial was degassed with argon before X-phos (518 mg, 1.09 mmol, Eq: 0.05) and tris(dibenzylideneacetone)dipalladium-chloroform adduct (562 mg, 543 μmol, Eq: 0.025) were added. The vial was closed and the reaction mixture was heated to 110° C. and stirred for 2 h. The reaction mixture was poured into 50 ml of water and extracted with EtOAc (3×50 ml). The organic layers were combined, dried over Na₂SO₄, filtered through sintered glass, concentrated and dried in vacuo. The crude material was purified by flash chromatography (silica gel, 120 g, 0% to 40% EtOAc in heptane). The fractions were combined, concentrated and dried in vacuo to afford the title compound methyl 4-amino-2′-chloro-4′-methyl-[1,1′-biphenyl]-3-carboxylate (5.08 g, 15.7 mmol, 72.3% yield) as brown solid. MS (ESI): 276.17 [M+H]+.

### b) 6-(2-Chloro-4-methylphenyl)-1H-benzo[c][1,2,6]thiadiazin-4(3H)-one 2,2-dioxide

To a solution of methyl 4-amino-2′-chloro-4′-methyl-[1,1′-biphenyl]-3-carboxylate (450 mg, 1.63 mmol, Eq: 1) in THF (12 ml) under argon was added sulfamoyl chloride (283 mg, 2.45 mmol, Eq: 1.5) at 10° C. The mixture was stirred for 5 h at room temp. At 0° C. aqueous NaOH solution (4.08 ml, 2M, 8.16 mmol, Eq: 5) was added. The reaction was diluted with 10 ml of water and extracted with EtOAc (3×20 ml). The organic layers were combined, dried over MgSO₄, filtered through sintered glass, concentrated and dried in vacuo. The reaction was diluted in diethylether, filtered through sartorius to obtain the title compound 6-(2-chloro-4-methylphenyl)-1H-benzo[c][1,2,6]thiadiazin-4(3H)-one 2,2-dioxide (403.5 mg, 1.23 mmol, 75.2% yield), MS (ESI): 323.08 [M+H]+.

### Example 2

### 6-(2-Chloro-4-methylphenyl)-1-(2-morpholinoethyl)-1H-benzo[c][1,2,6]thiadiazin-4(3H)-one 2,2-dioxide

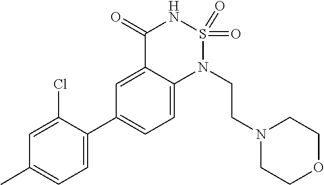 [SMILES: Cc1ccc(-c2ccc3c(c2)C(=O)NS(=O)(=O)N3CCN2CCOCC2)c(Cl)c1]

In a 10 ml round-bottomed flask, 6-(2-chloro-4-methylphenyl)-1H-benzo[c][1,2,6]thiadiazin-4(3H)-one 2,2-dioxide (40 mg, 124 μmol, Eq: 1) was combined with aqueous NaHCO₃ (2.5 ml). 4-(2-bromoethyl)morpholine (36.1 mg, 186 μmol, Eq: 1.5) was added and the reaction was stirred for 4 days at reflux. Every day another amount of 4-(2-bromoethyl)morpholine (36.1 mg, 186 μmol, Eq: 1.5) was added. The reaction mixture was diluted with 15 ml NaHCO₃ and extracted with EtOAc (3×20 ml). The organic layers were combined, dried over MgSO₄, filtered through sintered glass, concentrated and dried in vacuo. The crude material was purified by preparative HPLC to obtain 6-(2-chloro-4-methylphenyl)-1-(2-morpholinoethyl)-1H-benzo[c][1,2,6]thiadiazin-4(3H)-one 2,2-dioxide (4.3 mg, 7.95 μmol, 6.4% yield). MS (ESI): 436.3 [M+H]+.

### Example 3

### 6-(2-Chloro-4-methylphenyl)-1-(pyridin-4-ylmethyl)-1H-benzo[c][1,2,6]thiadiazin-4(3H)-one 2,2-dioxide

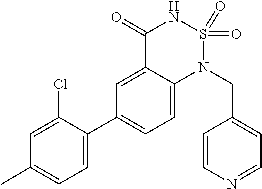 [SMILES: Cc1ccc(-c2ccc3c(c2)C(=O)NS(=O)(=O)N3Cc2ccncc2)c(Cl)c1]

In a 10 ml round-bottomed flask, 6-(2-chloro-4-methylphenyl)-1H-benzo[c][1,2,6]thiadiazin-4(3H)-one 2,2-dioxide (40 mg, 124 μmol, Eq: 1) was combined with aqueous NaHCO₃ (2.5 ml). 4-(chloromethyl)pyridine hydrochloride (30.5 mg, 186 Eq: 1.5) was added and the reaction was stirred for 2 h at reflux. The reaction mixture was diluted with 15 ml NaHCO₃ solution and extracted with EtOAc (3×20 ml). The organic layers were combined, dried over MgSO₄, filtered through sintered glass, concentrated and dried in vacuo to afford the title compound 6-(2-chloro-4-methylphenyl)-1-(pyridin-4-ylmethyl)-1H-benzo[c][1,2,6]thiadiazin-4(3H)-one 2,2-dioxide (13.6 mg, 30.9 μmol, 24.9% yield) as a white solid. MS (ESI): 412.0509 [M−H]−.

### Example 4

### 6-(2-Chloro-4-methylphenyl)-1-phenethyl-1H-benzo[c][1,2,6]thiadiazin-4(3H)-one 2,2-dioxide

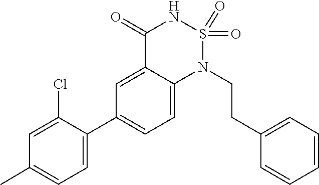 [SMILES: Cc1ccc(-c2ccc3c(c2)C(=O)NS(=O)(=O)N3CCc2ccccc2)c(Cl)c1]

In a 10 ml round-bottomed flask, 6-(2-chloro-4-methylphenyl)-1H-benzo[c][1,2,6]thiadiazin-4(3H)-one 2,2-dioxide (40 mg, 124 μmol, Eq: 1) was combined with aqueous NaHCO₃ solution (2.5 ml). (2-Bromoethyl)benzene (34.4 mg, 25.4 μl, 186 Eq: 1.5) was added and the reaction was stirred over night at reflux. The reaction mixture was diluted with 15 ml NaHCO₃ solution and extracted with EtOAc (3×20 ml). The organic layers were combined, dried over MgSO₄, filtered through sintered glass, concentrated and dried in vacuo to afford the title compound 6-(2-chloro-4-methylphenyl)-1-phenethyl-1H-benzo[c][1,2,6]thiadiazin-4(3H)-one 2,2-dioxide (10.2 mg, 23.4 μmol, 18.9% yield) as a white solid. MS (ESI): 425.0716 [M−H]−.

### Example 5

### 6-(2-Chloro-4-methylphenyl)-3-(2-morpholinoethyl)-1H-benzo[c][1,2,6]thiadiazin-4(3H)-one 2,2-dioxide

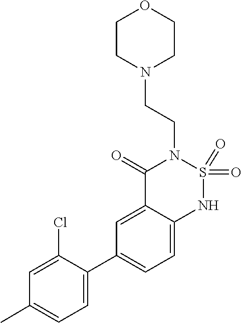 [SMILES: Cc1ccc(-c2ccc3c(c2)C(=O)N(CCN2CCOCC2)S(=O)(=O)N3)c(Cl)c1]

In a 10 ml round-bottomed flask, 6-(2-chloro-4-methylphenyl)-1H-benzo[c][1,2,6]thiadiazin-4(3H)-one 2,2-dioxide (40 mg, 124 μmol, Eq: 1) was combined with DMF (1 ml). 4-(2-Bromoethyl)morpholine (36.1 mg, 186 μmol, Eq: 1.5) was added and the reaction was stirred for 2 h at reflux. The reaction mixture was diluted with 15 ml NaHCO₃ solution and extracted with EtOAc. The organic layer was discarded. 2M HCl was added to the aqueous phase till the pH was acidic. Then it was extracted with EtOAc (3×20 ml). The organic layers were combined, dried over MgSO₄, filtered through sintered glass, concentrated and dried in vacuo. 27 mg of the dried material were further purified by preparative HPLC to afford the title compound 6-(2-chloro-4-methylphenyl) (2-morpholinoethyl)-1H-benzo[c][1,2,6]thiadiazin-4(3H)-one 2,2-dioxide (7 mg, 16.1 μmol, 13% yield) as a white solid. MS (ESI): 435.1022 [M−H]−.

### Example 6

### 1-Benzyl-6-(2-chloro-4-methylphenyl)-1H-benzo[c][1,2,6]thiadiazin-4(3H)-one 2,2-dioxide

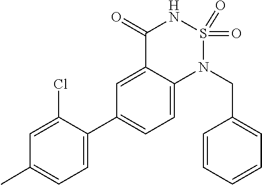 [SMILES: Cc1ccc(-c2ccc3c(c2)C(=O)NS(=O)(=O)N3Cc2ccccc2)c(Cl)c1]

In a 10 ml round-bottomed flask, 6-(2-chloro-4-methylphenyl)-1H-benzo[c][1,2,6]thiadiazin-4(3H)-one 2,2-dioxide (15 mg, 46.5 μmol, Eq: 1) was combined with aqueous NaHCO₃ (1 ml). Benzylbromide (11.9 mg, 8.28 μl, 69.7 μmol, Eq: 1.5) was added and the reaction was stirred for 2 h at reflux. The reaction mixture was diluted with 15 ml NaHCO₃ solution and extracted with EtOAc (3×20 ml). The organic layers were combined, dried over MgSO₄, filtered through sintered glass, concentrated and dried in vacuo to afford the title compound 1-benzyl-6-(2-chloro-4-methylphenyl)-1H-benzo[c][1,2,6]thiadiazin-4(3H)-one 2,2-dioxide (14 mg, 32.9 μmol, 70.8% yield) as a white solid. MS (ESI): 413.09 [M+H]+.

### Example 7

Malachite Green Assay to Measure cGAS Activity

Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 μM ATP (Sigma), 80 μM GTP (Sigma) and 100 nM Interferon Stimulating DNA (ISD) (Microsynth). Recombinantly expressed purified human cGAS (residues 161-522) was used at 25 nM.

All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4 U/mL PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.

All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.

The results of this assay are provided in Table 1. Table 1 provides IC50 values (04) for cGAS inhibition obtained for particular examples of the present invention as measured by the above-described assay.

| Example | IC50 cGAS (μM) |
|---|---|
| 1 | 2.94 |
| 2 | 9.6 |
| 3 | 14.23 |
| 4 | 17.21 |
| 5 | 17.58 |
| 6 | 20.1 |

### Example A

Film coated tablets containing the following ingredients can be manufactured in a conventional manner:

| Ingredients | Per tablet | |
|---|---|---|
| Kernel: | | |
| Compound of formula (I) | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titan dioxide | 0.8 mg | 1.6 mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 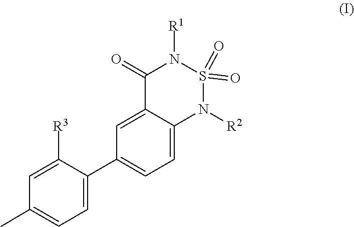
wherein
one of R¹ and R² is hydrogen and the other one is hydrogen, alkyl, morpholinylalkyl, pyridinylalkyl or phenylalkyl; and
R³ is halogen;
or a pharmaceutically acceptable salt or thereof.

2. A compound according to claim 1, wherein one of R¹ and R² is hydrogen and the other one is hydrogen or morpholinylalkyl.

3. A compound according to claim 1 or 2, wherein one of R¹ and R² is hydrogen and the other one is hydrogen or morpholinylethyl.

4. A compound according to any one of claims 1 to 3, wherein R³ is chlorine.

5. A compound according to any one of claims 1 to 4 selected from
6-(2-chloro-4-methylphenyl)-1H-benzo[c][1,2,6]thiadiazin-4(3H)-one 2,2-dioxide;
6-(2-chloro-4-methylphenyl)-1-(2-morpholinoethyl)-1H-benzo[c][1,2,6]thiadiazin-4(3H)-one 2,2-dioxide;
6-(2-chloro-4-methylphenyl)-1-(pyridin-4-ylmethyl)-1H-benzo[c][1,2,6]thiadiazin-4(3H)-one 2,2-dioxide;
6-(2-chloro-4-methylphenyl)-1-phenethyl-1H-benzo[c][1,2,6]thiadiazin-4(3H)-one 2,2-dioxide;
6-(2-chloro-4-methylphenyl)-3-(2-morpholinoethyl)-1H-benzo[c][1,2,6]thiadiazin-4(3H)-one 2,2-dioxide;
1-benzyl-6-(2-chloro-4-methylphenyl)-1H-benzo[c][1,2,6]thiadiazin-4(3H)-one 2,2-dioxide;
or a pharmaceutically acceptable salt thereof.

6. A compound according to any one of claims 1 to 6 selected from
6-(2-chloro-4-methylphenyl)-1H-benzo[c][1,2,6]thiadiazin-4(3H)-one 2,2-dioxide;
6-(2-chloro-4-methylphenyl)-1-(2-morpholinoethyl)-1H-benzo[c][1,2,6]thiadiazin-4(3H)-one 2,2-dioxide;
or a pharmaceutically acceptable salt thereof.

7. A process for the preparation of a compound according to any one of claims 1 to 6, comprising one of the following steps:
(a) the reaction of a compound of formula (A) 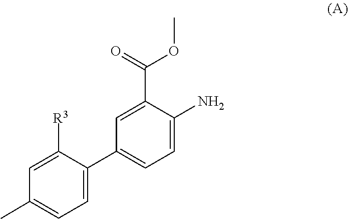
with H₂NSO₂Cl in a solvent and in the presence of an aqueous base; or
(b) the reaction of a compound of formula (B) 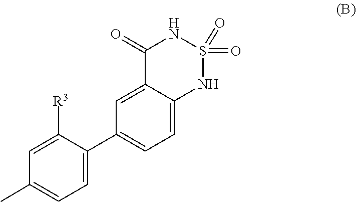
with R—X, optionally with a base, wherein R is R¹ or R² are as defined in any one of claims 1-6 and X is a leaving group.

8. A compound according to any one of claims 1 to 6, when manufactured according to a process of claim 7.

9. A compound according to any one of claims 1 to 6, for use as therapeutically active sub stance.

10. A pharmaceutical composition comprising a compound in accordance with any one of claims 1 to 6 and a therapeutically inert carrier.

11. The use of a compound according to any one of claims 1 to 6 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

12. The use of a compound according to any one of claims 1 to 6 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

13. A compound according to any one of claims 1 to 6 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

14. A method for the treatment of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound as defined in any one of claims 1 to 6 to a patient in need thereof.

15. The invention as hereinbefore described.
